(19) United States
(12) Patent Application Publication
(10) Pub. No.: US 2001/0053520 A1
(43) Pub. Date: Dec. 20, 2001

(54) Methods of making and using microarrays of biological materials

(76) Inventors: Carl Arne Krister Borrebaeck, Hjarup (SE); Roland Carlsson, Lund (SE)

Correspondence Address: PATREA L. PABST, HOLLAND & KNIGHT LLP, SUITE 2000, ONE ATLANTIC CENTER, 1201 WEST PEACHTREE STREET, N.E., ATLANTA, GA, 30309-3400, US

(21) Appl. No.: 09/811,075

(22) Filed: Mar. 16, 2001

Related U.S. Application Data
(60) Provisional application No. 60/192,256, filed on Mar. 27, 2000.

(30) Foreign Application Priority Data
Mar. 17, 2000 (GB) 0006425.3

(51) Int. Cl. C12Q 1/68

(52) U.S. Cl. 435/6

(57) ABSTRACT

The invention relates to methods of making arrays of anti-ligands for use in the analysis of ligands. Preferred ligands are proteins and preferred anti-ligands are antibodies, or antigen-binding variants or derivatives thereof. The microarrays of the invention provide a convenient means for analysing differential protein expression in cells.

The present invention relates to methods and materials for studying proteins in biological materials. In particular, the invention provides methods and materials for use in the study of differences in protein expression in samples of biological material, especially differences between diseased and normal tissue samples.

During the past few years developments within especially genomics e.g. the Human Genome Organisation Project (HUGO), have indicated the presence of a large number of structures as potential targets for therapeutic intervention. These kinds of studies have revealed information concerning gene sequences and the organisation of genes, and allowed deduction of primary structure of gene products. However, in much less than 50% of the completed gene sequences has any function been ascribed to the gene products.

The need for means to identify novel genes and gene products has brought about development of high throughput DNA and protein sequencing technologies. Thus, the speed of sequencing DNA, either genomic or cDNA, has increased dramatically so that today currently systems exist that can allow identity sequences at rates of 500 bp in twenty minutes. In addition, systems that target various aspects of DNA sequencing have developed. For some applications knowledge of the full length gene sequence is required, whereas for other applications only partial sequences are needed. Information on the presence or absence of defined mutations (SNP's) in particular genes is gathered together with information on whether or not a known gene is expressed or not, using sequence information as a specific tag for the gene in question. Also, bioinformatics systems that allow efficient handling of the enormous amount of data generated by the above methods have been developed.

Thus, the rapid development of technology in this field allows researchers to look at complex biological systems on a macroscopic rather than microscopic level. Presently, most macroscopic systems analyse gene expression, and especially differential gene expression. However, systems for the macroscopic analysis of gene products, proteins or poly-peptides, are less developed.

Information on how genes are expressed differentially is of importance when comparing e.g. pathologic (diseased) vs. normal tissue. The idea is that genes that are up- or down-regulated relate to the pathological state, and either their products, or proteins functionally linked to these products, may be used as target structures for therapeutic intervention. Recently, technologies based on DNA chip arrays, allowing analysis of tens of thousands of genes, have been developed and used successfully for characterisation of gene expression and for identification of differentially expressed genes (see refs 9, 24 and 40). Experiments that can be set up using this technology relate to, in addition to study of differences in gene expression between pathologic and normal tissue, study of what happens in cells and tissue after stimulation with biologically active compounds. For instance, signal transduction pathways that are activated after stimulation with a particular ligand may be identified, and effects on gene expression after stimulation with drugs or drug candidates may be discerned. These and similar tools have been met with great interest from the pharmaceutical and biotech industry.

However, it is gene products i.e. proteins and poly-peptides and not the genes themselves which exhibit function in a cell. Thus, proteins are the main target molecules for therapeutic interventions. Moreover, there is no direct relationship between gene activity, the level of mRNA and the level of protein encoded by the gene. Factors influencing mRNA and protein turnover can obliterate totally the relationship between levels of mRNA and protein. Consequently, mRNA-levels do not necessarily reflect the level of functional proteins in the cell or tissue (see refs 1, 3 and 23). In addition, proteins are often modified posttranslationally by e.g. glycosylation and phosphorylation. Such modifications greatly influence the activity of proteins. Whether or not a particular protein is modified or not can not be discerned from the mRNA or DNA sequence. Therefore, methods that allowed study, at a macroscopic or global level, of the expression of proteins, and, in addition, discriminate between modified forms of the proteins would be of great value. Such methods could permit simultaneous analysis of the proteome, ie the total protein content of a cell, or metabolome ie, the total content of molecules produced by metabolic processes.

Despite the potential benefits of methods of studying protein expression, the identification of proteins poses much greater problems than the identification of DNA. Even if the primary structure of a particular protein is known, knowledge of what molecules will specifically bind to the protein, and therefore serve as a probe for that protein, cannot be predicted. In contrast, when the sequence of a particular DNA or gene is known an oligo-nucleotide complimentary to, and specific for, that DNA can be made easily. Therefore, chip arrays based on DNA can be made and used for simultaneous detection of a multitude of genes or gene fragments without great difficulty.

Even though chip arrays for analysis of proteins have been described (see refs 7 and 10) their performance and use lags far behind that of DNA arrays.

Currently, the most common methods for macroscopic or global analysis of proteins are based on 2D-gel electrophoresis (see refs 29 and 14). This method has been in use for many years but has recently been developed into a system that meets some of the requirements for analysis at the global level (see ref 18). In addition, data analysis and handling techniques have improved significantly so that many proteins can be identified by position on a 2D-gel (see refs 18 and 32).

In order to identify proteins in spots on 2-D gels no direct method is available. Mass spectroscopy on protein eluted from identified spots is sometimes used to obtain a peptide sequence that can be used as a tag. The tag sequence can be compared to known sequences e.g. a database and, if it exhibits a high sequence identity with a known protein, a likely identity of the protein in that particular spot. However, such a system requires an elaborate set up of gel-electrophoresis, picture analysis, equipment for elution of protein spots, mass spectrometers, data handling and bioinformatics (see ref 30). Moreover, quantitative differences between spots are judged merely by the density of the spot and spots often overlap making it difficult to interpret the 2-D gel results.

The present invention provides methods and materials which overcome some of the limitations of 2D gel analysis. The invention permits the macroscopic or global analysis of protein in biological systems.

According to the invention there is provided a method of making an array of selected anti-ligands comprising:

(i) providing a library of anti-ligand molecules displayed for binding with a ligand on the surface of a replicable unit;

(ii) providing a mixture of ligands

(iii) exposing the library to the mixture whereby ligand/anti-ligand binding can take place;

(iv) isolating and amplifying the number of anti-ligands which bind ligands; and

(v) applying a preparation of the same anti-ligands, or a plurality of different anti-ligands, to a separate region of a substrate to form an array of separate anti-ligand-containing regions on a solid support.

In one embodiment of the invention the method comprises the further step of isolating ligands bound to anti-ligands on the surface of the replicable units between steps (iii) and (iv).

Conveniently, the ligands in the mixture are tagged by a tagging agent so that they can be isolated by an anti-tagging agent which binds to the tagging agent. The tagging agent is preferably biotin and the anti-tagging agent is avidin. However, skilled persons will appreciate that a variety of agents are available and suitable for use in the methods of the invention.

It is preferred that the mixture of ligands is separated on the basis of one or more parameters (eg size, electrical charge or isoelectric point) before it is exposed to the library. A preferred separation method is two-dimensional gel electrophoresis.

Advantageously, the ligands in the mixture are immobilised.

Advantageously, the ligands in the separated mixture are immobilised on a support surface, such as nitrocellulose or polyvinylidene diflouride (PVDF) membrane

It is most preferred that the separated ligand mixture is immobilised on a support surface which is a replica of the 2D gel and that the replica is used directly in step (iii) of the method of the invention. Such direct use of the gel replica has enormous time and cost advantages over methods which involve excision of individual ligand bands separately from a gel replica.

The anti-ligand preferably comprises a protein or polypeptide. Most preferably, the anti-ligand is an antibody or an antigen binding variant or derivative thereof. However, skilled persons will appreciate that anti-ligands can be provided from a variety of sources, especially molecular libraries such as antibody libraries (see refs 4 and 26), peptide libraries (see ref 36), expressed cDNA libraries (see ref 33), libraries on other scaffolds than the antibody framework such as affibodies (see ref 13) or libraries based on aptamers (see ref 21).

A particularly advantageous feature of the methods of the invention is that the identity of at least some, and preferably all, of the ligands and/or anti-ligands can be unknown. Hence, prior characterization of ligands, and/or anti-ligands is unnecessary.

It is most preferred that a plurality, especially 10 to 50 different anti-ligands are applied to each region of the substrate to form an anti-ligand array in step (vi) of the method of the invention. The use of a plurality of anti-ligands per region of the array confers enormous advantages over methods which involve the use of a single anti-ligand per region of an array.

Arrays produced by a method of the invention may find particular utility in a method according to a second aspect of the invention, comprising comparing the presence, absence and/or amount of one or more ligands in first and second biological samples by detecting differences in ligand/anti-ligand binding when an array is exposed to the samples.

Another embodiment of the invention relates to the use of two or more arrays of the invention in a method comprising comparing the presence, absence and/or amount of one or more ligands in first and second biological samples by detecting differences in ligand/anti-ligand binding when an array is exposed to the first biological sample and a substantially identical array is exposed to the second biological sample.

In a use according to the second aspect of the invention the ligands in the first and second biological samples are detectably labelled, preferably with different first and second fluorescent reporters so that, in use, during examination of the array under conditions of fluorescence excitation, anti-ligands in the array which are bound predominantly to ligands from one of the first and second biological samples give a first or second fluorescence emission; and anti-ligands which bind substantially equal numbers of ligands from the first and second biological samples give a combined fluorescence emission.

The first biological sample may be from a diseased cell type and the second biological sample may be from a corresponding cell type unaffected by the disease; or the first and second samples may be from the same cell type at rest and active respectively, making the uses of the invention advantageous in methods of studying protein expression and metabolite levels. The mixture of ligands provided in step (ii) of the method of the invention may be derived from the same source as the first or second biological sample. By derived from the same source we include the meaning that the mixture is, for example, from the same cell type.

Labelling of the ligand and anti-ligands with detectable labels other than fluorescent labels is also possible of course and well within the knowledge of skilled persons. The use of any detectable labels is intended to fall within the scope of the invention. In addition, the invention is intended to embrace other systems for detecting ligand/anti-ligand binding, including systems based on changes in electrical conducitivity or plasmone resonance, using for example nanoelectrodes (see WO 99/24823) or biosensors (see ref 25).

The first and second biological samples may be applied to identical but separate arrays of anti-ligands.

Definitions

By “ligand” we include the meaning of one member of a ligand/anti-ligand binding pair. The ligand may be, for example, one of the nucleic acid strands in a complementary, hybridized nucleic acid duplex binding pair; an effector molecule in an effector/receptor binding pair; or an antigen in an antigen/antibody or antigen/antibody fragment binding pair.

By “anti-ligand” we include the meaning of the opposite member of a ligand/anti-ligand binding pair. The anti-ligand may be the other of the nucleic acid strands in a complementary, hybridized nucleic acid duplex binding pair; the receptor molecule in an effector/receptor binding pair; or an antibody or antibody fragment molecule in antigen/antibody or antigen/antibody fragment binding pair, respectively.

The term “detectably labelled” as used herein is intended to encompass antigen (ligand) or antibody (anti-ligand) directly coupled to a detectable substance, such as a fluorescent dye, and antigen (ligand) or antibody (anti-ligand) coupled to a member of binding pair, such as biotin/streptavidin, or an epitope tag that can specifically interact with a molecule that can be detected, such as by producing a coloured substrate or fluorescence.

By “replicable unit” we include the meaning that the unit is capable of replication by itself or with the co-operation of one or more agents, for example, the replicable unit may be a bacteriophage which can infect a host cell and replicate itself. The replicable unit could also be a phagemid which is not capable of replication in a host cell by itself, but can replicate in the presence of a helper phage. Ribosome display units are also intended to fall within the scope of the term “replicable unit”.

By “array of regions on a solid support” we include the meaning of a linear or two-dimensional array of preferably discrete regions, each having a finite area, formed on the surface of a solid support. The solid support is typically glass or a polymer, the most commonly used polymers being cellulose, polyacrylamide, nylon, polystyrene, polyvinyl chloride or polypropylene. The solid supports may be in the form of tubes, beads, discs, silicon chips, microplates, polyvinylidene difluoride (PVDF) membrane, nitrocellulose membrane, nylon membrane, other porous membrane, non-porous membrane (eg plastic, polymer, perspex, silicon, amongst others), a plurality of polymeric pins, or a plurality of microtitre wells, or any other surface suitable for immobilising proteins and/or conducting an immunassay. The binding processes are well-known in the art and generally consist of cross-linking covalently binding or physically adsorbing the protein molecule to the solid support.

By “microarray” we include the meaning of an array of regions having a density of discrete regions of at least about 100/cm², and preferably at least about 1000/cm². The regions in a microarray have typical dimensions, e.g., diameters, in the range of between about 10-250 μm, and are separated from other regions in the array by about the same distance.

By “cell type” we include the meaning of a cell from a given source, e.g., a tissue, or organ, or a cell in a given state of differentation, or a cell associated with a given pathology of genetic makeup.

As used herein, the term “monoclonal antibody” shall be taken to include both an immunoglobulin molecule produced by an hybridoma, and an hybridoma producing one or more immunoglobulin molecules, irrespective of whether or not the specificity of said immunoglobulin molecules is the same. Monoclonal antibodies are obtainable by immunisation with an appropriate gene product, epitope, peptide, or fragment of a gene product, or alternatively, a mixture comprising a plurality of same. Monoclonal antibodies may be selected from naturally occurring polyclonal antibodies raised against one or more epitopes, peptides, or protein fragments, derived from recombinant or naturally-occuring sources.

To produce monoclonal antibodies, antibody producing cells (lymphocytes) can be harvested from an animal immunised with the protein and fused with myeloma cells by standard somatic cell fusion procedures, thus immortalizing these cells and yielding hybridoma cells. Such techniques are well known in the art (see, for example Douillard and Hoffman (1981). In: Compendium of Immunology Vol II (ed. Schwarz). For example, the hybridoma technique originally developed by Kohler and Milstein (1975) Nature 256: 495-499 as well as other techniques such as the human B-cell hybridoma technique (Kozbor et al. (1983) Immunol. Today 4:72), the EBV-hybridoma technique to produce human monoclonal antibodies (Cole et al (1985) In: Monoclonal antibodies in cancer therapy, Alan R Bliss Inc., pp 77-96), and screening of combinatorial antibody libraries (Huse et al. (1989) Science 246: 1275-1281). Hybridoma cells can be screened immunochemically for production of antibodies which are specifically reactive with the peptide and monoclonal antibodies isolated therefrom.

The term “antibody variant” shall be taken to refer to any synthetic antibodies, recombinant antibodies or antibody hybrids, such as but not limited to, a single-chain antibody molecule produced by phage-display of immunoglobulin light and/or heavy chain variable and/or constant regions, or other immunointeractive molecule capable of binding to an antigen in an immunoassay format that is known to those skilled in the art.

Recombinant antibodies, comprising immunoglobulin light and heavy chain variable and constant regions, expressed on the surface of a bacteriophage of virus particle, such as in a phage display library, are preferred. Optimization of high-affinity antibodies by phage display of combinatorial antibody libraries (Crosby W L, and Schoor P. (1995) Methods. Cell. Biol. 50:85; Winter J. (1994) Drug Dev. Res. 33:71) is a robust mimic of immune selection for natural antibody diversity, as an alternative to traditional and immunisation technologies. Human Fabs, single-chain antibodies (de Kruif J, Boel E, and Logtenberg T. (1995) J. Mol. Biol. 248:97; Deng S J, MacKenzie C R, Hirama T, Brousseau R, and Lowary T L, (1995) Proc. Natl. Acad. Sci. (USA) 92: 4992; Zdanov A, Li Y, Bundle D R, Deng S. J. and MacKenzie R (1994) Proc. Natl. Acad. Sci (USA) 91:6423), or disulfide-stabilized Fv's (Brinkman U, Chowdhury P S, Roscoe D M, and Pastan I. (1995) J. Immunol. Methods 182:41) can be isolated with specificities against virtually any targeted antigen, either foreign or self (Ditzel H J, and Burton D R. (1995) In: Vaccine 95: Mol. Approaches Control Infect. Dis. Annu. Meet., 12^{th} (ed. R M Chanock), Cold Spring Harbor, N.Y.: Cold Spring Harbor Lab. Press. pp19 et seq.), hapten (Short M K, Jeffrey P D, Kwong R-F, and Margolies M N (1995) J. Biol. Chem. 270;28541), carbohydrate (Deng S J, MacKenzie C R, Hirama T, Brousseau R, and Lowary T L, (1995) Proc. Natl. Acad. Sci. (USA) 92: 4992; Zdanov A, Li Y, Bundle D R, Deng S. J. and MacKenzie R (1994) Proc. Natl. Acad. Sci (USA) 91:6423), protein, DNA (Barbas S M, Ditzel H J, Salonen E M, Yang W P, and Silverman G J. (1995) Proc. Natl. Acad. Sci. (USA) 92:2529), or RNA (Powers J E, Marchbank M T, and Deutscher S L. (1995) Symp. RNA Biology I. RNA-Protein Interactions, In: Nucleic Acids Symp. Series: 33, pp240). Moreover, cell sub-population-specific monoclonal antibodies may also be derived from synthetic phage antibody libraries (de Kruif J, Boel E, and Logtenberg T. (1995) J. Mol. Biol. 248:97). Techniques for the production of recombinant antibodies in phage display libraries are well-known in the art (Crosby W L, and Schorr P (1995) Methods. Cell. Biol. 50:85; Winter J. (1994) Drug Dev. Res. 33:71).

Several bacteriophage-based vector systems are available for expressing immunoglobulins in phage display libraries, for example the λlmmunoZAP vector series including λlmmunoZAP L, λlmmunoZAP H, λlmmunoZAP H/L and λSurfZAP, for expressing immunoglobulin Fab fragments, light chains and heavy chains on the surface of a filamentous phage (Hogrefe H. H., Mullinax R. L., Lovejoy A. E., Hay B. N., and Sorge J. A. (1993) Gene 128:119-126; Moulinax R. L., Gross E. A., Amberg J. R., Hay B. N., Hogrefe H. H., Kubitz M. M., Greener A., Alting-Mees M., Ardourel D., Short J. M., Sorge J. A., and Shopes B.; (1990) Proc. Natl. Acad. Sci (USA) 87: 8095-8099., 1990; Shopes B. (1992) Third Annual IBC Int. Conf. Antibody Engineering 0:121-131; Stratagene, Calif., USA).

The term “antibody derivative” refers to any modified antibody molecule that is capable of binding to an antigen in an immunoassay format that is known to those skilled in the art, such as a fragment of an antibody (Fab fragment), or an antibody molecule that is modified by the addition of one or more amino acids or other molecules to facilitate coupling the antibodies to another peptide or polypeptide, to a large carrier protein or to a solid support (eg. the amino acids tyrosine, lysine, glutamic acid, aspartic acid, cysteine and derivatives thereof, NH₂-acetyl groups or COOH-terminal amido groups, amongst others).

Preferably, the antibody molecules or antibody variants or derivatives used in the methods of the invention are detectably labelled, and preferably modified by the addition of one or more reporter molecules, which are bound thereto to facilitate their detection.

According to this embodiment, it is possible to detect one or more antigenic determinants in a labelled mixture of antigens, by a variety of means including fluorescence, chemiluminescence, isotopic determination, enzymatic labelling, amongst others.

The term “detectably labelled” as used herein is intended to encompass antigen (ligand) or antibody (anti-ligand) directly coupled to a detectable substance, such as a fluorescent dye, and antigen (ligand) or antibody (anti-ligand) coupled to a member of binding pair, such as biotin/streptavidin, or an epitope tag that can specifically interact with a molecule that can be detected, such as by producing a coloured substrate or fluorescence.

Substances suitable for detectably labeling proteins include fluorescent dyes such as fluorescein isothiocyanate (FITC), fluorescein, rhodamine, tetramethyl-rhodamine-5-(and 6)-isothiocyanate (TRITC), Texas red, cyanine dyes (Cy3 and Cy5, for example), and the like; and enzymes that react with colorometric sustrates such as horseradish peroxidase. The use of fluorescent dyes is generally preferred in the practice of the invention as they can be detected at very low amounts.

Preferred reporter molecules for binding covalently or non-covalently to antibodies include, but are not limited to, radiochemicals, fluorescent compounds such as rhodamine, biotin, DIG, immunologically-interactive peptides such as FLAG peptides, poly-Hil or poly-Lys amino acid sequences or other known amino acid string, protein A, lectins (eg phytohemagglutinin A), secondary antibodies and functional enyzmes, such as alkaline phosphatase, horseradish peroxidase, the Escherichia coli β-galactosidase enzyme, the firefly luciferase protein (Ow, D. W.: Wood, K. V.; DeLuca, M.; de Wet, L. R.; Helinski, D. R.; and Llowell, S. (1986) Science 234:856-859; Thompson et at, 1991) and the green fluorescent protein (Prasher, D. C. et al (1992) Gene 111:229-233; Chalfie, M. et al (1994) Science 263:802-805; Inouye, S., and Tsuji, F. I. (1994) FEBS Letts 341:227-280; Cormack, B, et at (1996) Gene (in press); Haas, J. et al (1996) Curr. Biol. 6:315-324; see also GenBank Accession No. U55762).

In the case of reporter molecules comprising immunologically-interactive peptides or functional enzymes, these are preferably linked to recombinant antibodies by producing fusion proteins comprising both the immunoglobulin and reporter molecule moieties, wherein the corresponding gene regions encoding said moieties are spliced together in-frame and the recombinant nucleic acid molecule is expressed in a suitable cellular, viral or bacteriophage expression system. Techniques for producing such fusions molecules are well-known in the art.

The antibodies and/or antibody variants and/or antibody derivatives used in the methods of the invention may be derived from monoclonal or polyclonal antibody sources. For example, nucleotide sequences encoding immunoglobulin variable light and heavy chains may be derived from hybridomas or lymphocytes derived from animals that have been previously immunised with antigen, as for the production of monoclonal or polyclonal antibodies. Conventional immunisation strategies may be utilised to facilitate polyclonal or monoclonal antibody production, or the production of antibody derivatives, including the immunisation of animals, in particular rabbits or mice, with: whole cells, tissues, organs or whole organisms or sub-cellular fractions or lysates, extracts or other derivatives thereof; and/or DNA derived from biological samples corresponding to the whole or fractions of cells, tissues, or organisms and lysates thereof; (ie DNA vaccination); and/or proteins derived from a biological sample that has been subjected to a separative procedure, such as 2-dimensional gel eletrophoresis; and/or synthetic or recombinant peptides comprising signature amino acid sequences; and/or a “soup” comprising ligand mixture.

Molecular libraries such as antibody libraries (see refs 4 and 26), peptide libraries (see ref 36), expressed cDNA libraries (see ref 33), libraries on other scaffolds than the antibody framework such as affibodies (see ref 13) or libraries based on aptamers (see ref 21) may be used as a source from which binders (anti-ligands), or probes that are specific for a large multitude of proteins, poly-peptides or post-translationally modified proteins or poly-peptides are preferably selected for use in the methods of the invention. The molecular libraries may be expressed in vivo in prokaryotic (see refs 4 and 26) or eukaryotic (see ref 22) cells or may be expressed in vitro without involvement of cells (see refs 15, 16 and 28). In cases when protein based libraries are used often the genes encoding the libraries of potential binders (anti-ligands) are packaged in viruses and the potential binder (anti-ligand) is displayed at the surface of the virus (see refs 4, 26 and 36). The most commonly used such system, today, is filamentous bacteriophage displaying antibody fragments at their surfaces, the antibody fragments being expressed as a fusion to the minor coat protein of the bacteriophage (see refs 4 and 26). However, also other systems for display using other viruses (EP 39578), bacteria (see refs 13, 5 and 6), and yeast (see ref 35) have been used. In addition, recently, display systems utilising linkage of the poly-peptide product to its encoding mRNA in so called ribosome display systems (see refs 15, 16 and 28), or alternatively linkage of the polypeptide product to the encoding DNA (see U.S. Pat. No. 5,856,090 and WO 98/37186) have been presented.

When potential binders (anti-ligands) are selected from libraries one or a few known selector (ligand) molecules are usually employed. Even when purified selector molecules (ligands) cannot be used, as may be the case when cell-bound antigens are used, selections and analyses are set up so that the specificity of the binders (anti-ligand) for a defined selector (ligand) molecule can be ascertained. When dealing with thousands of selector molecules (ligands) keeping track of the relationship between selector (ligand) and specific binder (anti-ligand) then becomes a prohibitive problem as well as the mere selection of these specific anti-ligands against thousands of ligands. However, use of array technologies and systems for analyses of binding to spots ordered in arrays can eliminate this problem. Depending on the available technology the solution will be somewhat different.

If technology were available to generate and analyse an unlimited number of spots all potential binders (anti-ligands) e.g. antibody fragments in a library could be cloned and spotted in an array format. Next, antigens (ligands) from a defined source would be allowed to bind to the array. To spots that happened to contain binders (anti-ligands) against a particular antigen (ligand), that particular antigen (ligand) would bind, and binding would give a readable signal. Analysis of the binding would give a pattern that could be compared to the pattern obtained after a different preparation of antigens (ligands) was subjected to an identical array. The two antigen (ligand) preparations could e.g. represent pathological and normal cells or tissues, respectively. Differences in pattern would then correspond to antigens (ligands) that were differentially expressed, or posttranslationally modified, in the two cells or tissues. Importantly, the knowledge of the identity and specificity of each e.g. antibody clone (anti-ligand) would not be a prerequisite for generation of meaningful information since the mere difference in pattern and signal topology on the array would give information on which spots that were of interest. If needed, the identity of these clones (anti-ligands) and their corresponding antigens (ligands) may be determined in a subsequent step, using e.g. mass spectrometry. Today, technology that allows this approach is not at hand. Hence, alternative methods need to be developed in order to reduce library size, and thus, the number of spots in a meaningful way.

One way to reduce library size is to perform selections against antigens (ligands) obtained from cells or tissues of interest. However, in congruence with the above there is no need to acquire knowledge of the specificity of individual clones (anti-ligands), nor the identity of the antigens (ligands) used for selection. Clones of binders (anti-ligands), based on e.g. antibody fragments and isolated from the library, may be spotted onto the array, and antigens (ligands) from the tissue of interest will then be subjected to the array. Binding will give a readable topology, and this topological pattern may then be compared to the pattern generated by antigens (ligands) obtained from a source that is to be compared to the first source. Again difference in pattern will indicate which spots that contain antigens (ligands) that are differentially expressed. To further reduce the number of spots in the array a limited number of library clones (anti-ligands) can be mixed and applied in the same spot. There is no need that the clones (anti-ligands) must bind to the same antigen (ligand). The reason for this is that most antigens (ligands) are kept constant even between cells of different functional state. Thus, the fraction of antigens (ligands) that are expected to be differentially expressed is rather low.

Hence, the risk that specificities of the binders (anti-ligands) in any single spot should be directed to two differentially expressed antigens is low, even if a limited number of binders (anti-ligands) are present in each spot. Consequently, a differentially expressed antigen (ligand) will most probably be detected using this type of array.

In these type of arrays the position of the spots is of no real importance and will give no information as to the identity of the bound antigens (ligands). However, if antigens (ligands) are separated, by any means, prior to them being used for selection of binders (anti-ligands) from the molecular library, and the selected binders (anti-ligands) are spotted in relation to the separated antigens (ligands), information on identity of the spots will be available. Any means of separation could be used, but in order to facilitate selection of binders (anti-ligands) the separated antigens (ligands) should be possible to handle simultaneously. A feasible means of separation is to use 2-D gel electrophoresis. After separation, the antigens (ligands) may be blotted onto a membrane e.g. a nitrocellulose filter and optionally re-natured. Members (anti-ligands) from the molecular library will then be allowed to bind to the separated antigens. In a next step non-bound members will be washed away and bound members (anti-ligands) will be eluted and amplified. This procedure may be repeated after having amplified the bound library members (anti-ligands) for any number of cycles deemed necessary. Finally, the membrane containing the specifically bound library members (anti-ligands) will be divided into defined sections e.g. 100×100 and the binders (anti-ligands) amplified separately. Binders (anti-ligands) will then be spotted onto an array and a particular spot will, thus, comprise binders (anti-ligands) against antigens (ligands) found at a particular location after separation using e.g. 2-D gel electrophoresis. A spot that lights up in the array analysis will now have an identity that is defined by the separation method used. Using this procedure it is also likely that each spot will contain several clones of binders (anti-ligands) towards the same antigen (ligand). This will have important implications for the detection and analysis step since the affinity of each binder will be less important and the antigen will be bound, by several clones, with high avidity. Thus an approach using pre-separation of antigens is preferred over approaches where no such pre-separation is employed.

The formation of antibody:antigen complexes can be performed under a variety of conditions to identify antibodies with varying binding characteristics. Antigen-containing reaction solutions can contain varying degrees of salt or be conducted at varying pH levels. In addition, the binding reaction can be carried out at varying temperatures. Each set of conditions will identify antibodies with different affinity for the antigen. For example, antibodies that bind at pH 2 may have utility under highly acidic conditions such as those that exist in the stomach. Similarly, antibodies that bind at temperatures near boiling may be useful in studying thermophilic organisms. In general pH conditions will range from 2-10 (most preferably around pH 8), temperatures from 0° C.-100° C. and salt conditions from 1 μM to 5 M (in the case of NaCl).

Affinity constants are a measure of the interaction between a particular ligand and its cognate receptor. The “binding affinity” or the measure of the strength of association between a particular antibody:antigen interaction is generally measured by affinity constants for the equilibrium concentrations of associated and dissociated configurations of the antibody and its antigen. Preferably the binding of the antigen should occur at an affinity of about Kₐ=10⁻⁶ M or greater to be useful for the present invention, with greater than about 10⁻⁷ M being more preferable, and most preferably between about 10⁻⁸ M and about 10⁻¹¹ M. Antibody fragments will generally have affinities in the range of about 10⁻⁷ M to 10⁻⁸ M.

Analysis of molecules bound to the array spots can be quantitative, semi-quantitative or qualitative. Most analyses are semi-quantitative and many of them are based on readout systems depending on labelled ligands. The label is often a fluorophore or a radioisotope. Fluorophores are used successfully in DNA arrays (available from Affymetrix, Inc. CA) and allow rapid analysis of large numbers of spots. In some protein chips with rather few spots mass spectroscopy is used for analysis (see ref 7), and then a sequence tag can be obtained that can be used for identification of the bound component. Although mass spectrometry does not require any labels this technology cannot be used, at least not today, for analysis of large arrays with many samples. In contrast, in addition to read out systems based on fluorescence novel developments based on changes in electrical conductivity or plasmone resonance can be applied for analyses of bound components, using e.g. nanoelectrodes (see WO 99/24823) or biosensor technology (see ref 25).

In another embodiment of the invention, microarrays of uncharacterized antibodies are used to compare the protein expression profiles of cells, for example, comparisons can be made between a population of cells from one tissue, and a second tissue, or from cells derived from a particular tissue but from different species. Comparisons can be made between normal cells and cells from the same tissue type that originate from an individual with a pathogenic disorder. For example, comparisons can be made between normal cells and cancer cells. Comparisons can additionally be made between cells in a resting state and cells in an activated state.

In another example, the disclosed arrays are useful for evaluating the expression of proteins by pathogens, such as, for example, bacteria, parasites, viruses, and the like. A solution (such as a lysate) made from the pathogen which represents all proteins expressed by the pathogen can be used to contact an antibody array to proteins. These antibodies have utility as diagnostic agents as well as potential therapeutics.

Microarrays and the methods disclosed herein can be used in methods of diagnosing particular disorders. For example, a collection of antibodies specific for a range of antigens associated with one or more disorders can be arrayed and contacted with a bodily fluid containing antigens whose presence, or absence, would indicate a particular disorder. The advantage of using a microarray over a conventional immunoassay is the ability to include a population of antibodies diagnostic for a variety of disorders on a single surface, significantly reducing time, costs and materials needed to effect a diagnosis.

For example, if a patient presents with symptoms that are characteristic of several distinct disorders which can be distinguished on the basis of the presence or absence of one or more proteins, a single microarray assay could be used to make a specific diagnosis, thus allowing the patient to be properly treated.

Preferred non-limiting examples which embody certain aspects of the invention will now be described.

### EXAMPLE 1

Arrays of selected anti-ligands without knowledge of ligand identity or relationship between anti-ligand and ligand.

Anti-ligand library

In this and the following example the potential binders (anti-ligands) are members of an antibody fragment library displayed as fusions to the minor coat protein of filamentous phage (see U.S. Pat. No. 5,969,108 and refs 2 and 27). The library may be based on antibody fragments from naive or immunised animals or humans (see refs 8, 39 and 34). It may also be based on antibody fragments that have been made from synthetic genes (see refs 17 and 12), from artificially rearranged genes (U.S. Pat. No. 5,969,108) or from antibody genes obtained after CDR-shuffling (see ref 19).

Ligand mixture

An antigen (ligands) preparation of choice e.g. from tumour tissue, that may be a complex mixture of antigens may be immobilised to a solid phase e.g. immunotubes or be detectably labelled eg, biotinylated (see ref 27).

Exposure of ligands to anti-ligands

A preparation of a library of phage displayed antibodies is then subjected to the antigens and allowed to bind to the antigens. In case biotinylated antigen is used, phage bound to antigen is retrieved by use of avidin coated beads that may be magnetic. Non-bound phage are then washed away. Suitable washing conditions are known to those skilled in the art (see, for example, Ausbel, et al., Short Protocols in Molecular Biology, 3^{rd} Ed., 1995).

Application of ligand-bound replicable particles

The bound phage are eluted from the antigens using standard technologies and allowed to infect pili-expressing E. coli in order to expand the number of bound and eluted phage (see ref 27). After infection with a helper phage e.g. M13K07 (see ref 27) or R408 (see ref 11) phage particles are recovered. This phage preparation may now be subjected to the immobilised antigen preparation and the selection and expansion round repeated. Typically 2 to 4 selection and expansion rounds using standard technology is used (see ref 27).

Isolation of selected anti-ligands

In a next step the selected phage are allowed to infect E. coli and are cloned to allow production of soluble antibody fragments. This can be done either by use of an amber stop between the antibody fragment and the phage coat protein after infection of a non-supressor E. coli strain (see ref 26) or by transfer of the gene encoding the antibody fragments from the phage display vector to an expression vector. Random clones are then picked and expanded to allow production of soluble antibody fragments. The antibodies from the clones may then be checked for cross-reactivity to irrelevant antigens using standard technologies (see ref 10).

Production and use of selected anti-ligand array

The antibodies (anti-ligands) are then applied to an array at one clone per spot using standard technology. Methods and apparatus for fabricating microarrays of biological samples are also described in WO 95/35505, the disclosure of which is incorporated herein by reference.

Methods for creating microarrays are known in the art including printing on a solid surface using pins (passive pins, quill pins, and the like) or spotting with individual drops of solution. Passive pins draw up enough sample to dispense a single spot. Quill pins draw up enough liquid to dispense multiple spots. Bubble printers use a loop to capture a small volume which is dispensed by pushing a rod through the loop. Microdispensing uses a syringe mechanism to delivery multiple spots of a fixed volume. In addition, solid supports, can be arrayed using pizoelectric (ink jet) technology, which actively transfers samples to a solid support.

One method is described in Shalon and Brown (WO 95/35505, published Dec. 28, 1995) which is incorporated herein by reference in its entirety. The method and apparatus described in Shalon and Brown can create an array of up to six hundred spots per square centimeter on a glass slide using a volume of 0.01 to 100 nl per spot. Suitable concentrations of antibody range from about 1 ng/μl to about 1 μg/ul.

Other methods of creating arrays are known in the art, including photolithographic printing (Pease, et al, PNAS 91(11):5022-5026, 1994) and in situ synthesis.

The microarrays can be created on a variety of solid surfaces such as plastics (eg, polycarbonate), a complex carbohydrates (eg, agarose and sepharose), acrylic resins (eg. polyacrylamide and latex beads), and nitrocellulose. Preferred solid support materials include glass slides, silicon wafers, and positively charged nylon.

Methods for covalent attachment of antibodies to a solid support are known in the art. Examples of such are found in Bhatia, et al, Anal. Biochem 178(2): 408-413, 1989; Ahluwalia, et al, Biosens. Bioelectron. 7(3):207-214, 1992; Jonsson, et al, Biomaterials 17(22):2199-2207, 1996, all of which are incorporated by reference herein in their entirety.

Methods of reducing non-specific binding to a solid surface are well known in the art and include washing the arrayed solid surface with bovine serum albumin (BSA), reconstituted non-fat mile, salmon sperm DNA, porcine heparin, and the like (see Ausubel, et al, Short Protocols in Molecular Biology, 3^{rd} ed. 1995).

Antigen preparation of the type used for selection of the phage bound antibody fragments is then subjected to the array and antigens will bind to their corresponding antibodies on the array.

Detection of ligand/anti-ligand binding

Bound antigens may then be detected using a number of detection technologies including plasmone resonance, electrical conductivity and, in case the antigen preparation was detectably labelled fluorescence or radioactivity. To get informative data another antigen preparation from the normal counterpart to the tumour tissue is then subjected to an identical array as was used for the tumour tissue. Spots that differ between the two tissues represent antigens that are differentially expressed in the two tissues and therefore constitute interesting candidates for further analysis.

### EXAMPLE 2

Arrays of selected anti-ligands without knowledge of ligand identity or relationship between ligand and anti-ligand and containing several unknown clones of anti-ligands per spot.

The experimental setup is identical as that of Example 1 except that antibody fragments from several random clones are pooled and applied to the array in the same spot. Preferably more than 10 but less than 50 clones are pooled for each spot. This approach will allow many more antibody clones to be applied to an array than is the case if only one clone is applied to each spot. The identity of the clones is unknown and the probability that the clones in any given spot will be directed to the same antigen is low. Thus comparative analysis of any given spot has a potential to detect differential expression of a number of antigens. The possibility that the different antibodies in any given spot should be directed to two or more differentially expressed antigens is low when the number of antibody clones is kept between 10 and 50. In addition, there is also a possibility that any cloned antibody specificity may be represented in several pools and thus end up in several spots on the array and this may result in some redundancy in information.

### EXAMPLE 3

Array of selected anti-ligands spotted in relation to pattern obtained after separation of ligands.

Antigens (ligands) in antigen preparations may be separated on the basis of differences in e.g. size, electrical charge or isoelectric point using e.g. size exclusion chromatography (see ref 31), capillary electrophoresis (see ref 20), gel electrophoresis or isoelectric focusing, or combinations of these as in 2D-gel electrophoresis (see refs 29 and 14). Separation of complex mixtures of antigens utilising any of the above technologies will give information on identity of the antigens as to have a certain size, isoelectric point, electrophoretic mobility e.t.c., and this information is of value in the further identification of an antigen of interest. Thus, when an antigen preparation obtained from e.g. a tumour tissue is separated employing e.g. 2D-gel electrophoresis the position of the antigen on the gel will reveal much of its identity, and, in fact, many spots on 2D-gels have been identified using additional analytical methods as e.g. mass spectrometry.

When the separated antigens (ligands), in the format obtained after separation, are used for selection of anti-ligands from e.g. phage display antibody library, anti-ligands specific for the separated antigens (ligands) will be possible to obtain. Such a process, by way of example, will be illustrated below.

Separation of ligands in the ligand mixture

An antigen (ligand) preparation obtained from a tumour tissue is separated using 2D-gel electrophoresis. The separated antigens are then blotted onto protein binding membranes such as nitrocellulose or PVDF membranes while keeping their relative positions. The membrane thus becomes a replica of the gel. After blotting residual binding sites on the membrane are blocked using standard technologies (see refs 37 and 38).

Exposure to anti-ligand library

The blocking agent is preferably added to the anti-ligand preparation prior to mixture with the immobilised ligands to prevent binding of anti-ligands to the blocking agent on the gel replica. In a next step a phage display antibody fragment library is subjected to the gel replica membrane in a suitable volume and incubated for e.g. two hours under gentle rocking or agitation. Phage displaying antibody fragments (anti-ligands) will now bind to their corresponding antigens (ligands) immobilised to the gel replica membrane after which un-bound phage are washed away. Bound phage are then eluted from the gel replica membrane using e.g. low pH or trypsin (see U.S. Pat. No. 5,969,108). The retrieved phage are then allowed to infect E. coli and a new phage preparation is made after infection of these bacteria with a helper phage in case a phagemid system is used (see U.S. Pat. No. 5,969,108). If a phage vector is used no helper phage are needed. The resulting phage preparation is then used for reselection on a new membrane which is a replica of a 2D-gel obtained in the same way as the former one. The above selection and amplification process is then repeated two or three times. After the last selection the gel replica membrane is sectioned and bound phage are eluted from each section separately.

Amplification of selected anti-ligands

Each preparation of phage, corresponding to a particular section of the gel replica membrane, are amplified separately and soluble antibody fragments (anti-ligands) are produced from each preparation.

Preparation and use of array of selected anti-ligands

The antibodies (anti-ligands) from each preparation are then spotted onto an array keeping track of the origin of the antibody preparation in relation to the position on the 2D-gel. Identical arrays are made from the same antibody fragment preparations. These arrays are then used to analyse differential expression of antigen (ligand) between tumour tissue and normal tissue. To do this antigen preparations from the two tissues are made under identical conditions and allowed to bind identical arrays respectively. Bound antigens are then analysed using e.g. plasmone resonance or fluorescence (in case the antigens had been labelled with a fluorochrome). Differences in signal pattern indicate differences in antigen expression between the two tissues and the position on the array of the differing spots indicate the identity of the antigen in relation to the 2D-gel.

In any one of the above applications of the methods and materials of the invention the genetic information for the antibodies applied in each spot is kept separated in bacteria. Therefore, when a particular spot has been identified specific reagents for the particular antigens, or proteins, that have bound to the spot are easily available. These antibodies may be used for further analysis of the identified proteins. In case the antigen in question exhibits potential as a target for therapeutic intervention, antibodies that may be developed rapidly into therapeutic agents are already at hand.

The methods of the present invention provide a way to study variation in protein content globally involving chip or array technologies. The advantages of such technologies as compared to e.g. 2-D gel electrophoresis based technologies are numerous and include increased speed, sensitivity, reproducibility, and number of distinct regions or spots that may be analysed. A major problem which the invention solves in order to make this feasible is how to generate a large enough number of probes that can be used for array based identification of, in principle, tens of thousands of variant proteins or poly-peptides. The probes also have the potential to discriminate between post-translationally modified variants of proteins or poly-peptides caused by e.g. differential glycosylation or phosphorylation. The present invention demonstrates a way to solve these problems and how to use the invention for building a system that permits global analysis of protein content in biological systems.

We claim:

1. A method of making an array of selected anti-ligands comprising:
(i) providing a library of anti-ligand molecules displayed for binding with a ligand on the surface of a replicable unit;
(ii) providing a mixture of ligands;
(iii) exposing the library to the mixture whereby ligand/anti-ligand binding can take place;
(iv) isolating and amplifying the number of anti-ligands which bind ligands; and
(v) applying a preparation of the same anti-ligands, or a plurality of different anti-ligands, to a separate region of a substrate to form an array of separate anti-ligand-containing regions on a solid support.

2. A method as claimed in claim 1, further comprising a step of isolating ligands bound to anti-ligands on the surface of the replicable units between steps (iii) and (iv).

3. A method as claimed in claim 1 wherein the ligands in the mixture are immobilised.

4. A method as claimed in claim 1 wherein the mixture of ligands is separated on the basis of one or more parameters before it is exposed to the library.

5. A method as claimed in claim 4 wherein the mixture of ligands is separated using two-dimensional gel electrophoresis.

6. A method as claimed in claim 5 wherein the ligands in the separated mixture are immobilised on a support surface.

7. A method as claimed in claim 6 wherein the support surface is a nitrocellulose or polyvinylidene difluoride (PVDF) membrane.

8. A method as claimed in claim 6 wherein the support surface is a replica of the two-dimensional gel and is used directly in step (iii) of the method of claim 1.

9. A method as claimed in claim 2 wherein the ligands in the mixture are tagged by a tagging agent so that they can be isolated by an anti-tagging agent which binds to the tagging agent.

10. A method as claimed in claim 9 wherein the tagging agent is biotin and the anti-tagging agent is avidin.

11. A method as claimed in claim 1 wherein the anti-ligand comprises a protein or polypeptide.

12. A method as claimed in claim 11 wherein the anti-ligand is an antibody or an antigen binding variant or derivative thereof.

13. A method as claimed in claim 1 wherein the anti-ligand is a nucleic acid.

14. A method as claimed in claim 1 wherein the identity of at least some of the ligands and/or anti-ligands is unknown.

15. A method as claimed in claim 14 wherein the identity of substantially all of the ligands and/or anti-ligands is unknown.

16. A method as claimed in claim 1 wherein from 10 to 50 different anti-ligands are applied per region of the array.

17. Use of an array obtainable by a method as claimed in claim 1 in a method comprising comparing the presence, absence and/or amount of one or more ligands in first and second biological samples by detecting differences in ligand/anti-ligand binding when the array is exposed to the samples.

18. Use of two or more substantially identical arrays obtainable by a method as claimed in claim 1 in a method comprising comparing the presence, absence and/or amount of one or more ligands in first and second biological samples by detecting differences in ligand/anti-ligand binding when an array is exposed to the first biological sample and a substantially identical array is exposed to the second biological sample.

19. A use as claimed in claim 17 wherein the ligands in the first and second biological samples are labelled with different first and second fluorescent reporters so that, in use, under examination of the array under conditions of fluorescence excitation, anti-ligands in the array which are bound predominantly to ligands from one of the first and second biological samples give a first or second fluorescence emission; and anti-ligands which bind substantially equal numbers of ligands from the first and second biological samples give a combined fluorescence emission.

20. A use as claimed in claim 18 wherein the ligands in the first and second biological samples are labelled with different first and second fluorescent reporters so that, in use, under examination of the array under conditions of fluorescence excitation, anti-ligands in the array which are bound predominantly to ligands from one of the first and second biological samples give a first or second fluorescence emission; and anti-ligands which bind substantially equal numbers of ligands from the first and second biological samples give a combined fluorescence emission.

21. A use as claimed in claim 17 wherein the first and second biological samples are applied to identical but separate arrays of anti-ligands.

22. A use as claimed in claim 18 wherein the first and second biological samples are applied to identical but separate arrays of anti-ligands.

23. A use as claimed in claim 17 wherein the mixture of ligands provided in step (ii) of the method of making the array is derived from the same source as the first or second biological sample.

24. A use as claimed in claim 18 wherein the mixture of ligands provided in step (ii) of the method of making the array is derived from the same source as the first or second biological sample.

25. A use as claimed in claim 17 wherein the first biological sample is from a diseased cell type and the second biological sample is from a corresponding cell type unaffected by the disease.

26. A use as claimed in claim 18 wherein the first biological sample is from a diseased cell type and the second biological sample is from a corresponding cell type unaffected by the disease.
